(19) United States
(12) Patent Application Publication
(10) Pub. No.: US 2004/0024292 A1
(43) Pub. Date: Feb. 5, 2004

(54) System and method for assigning a computer aided detection application to a digital image

(75) Inventors: Wido Menhardt, Los Gatos, CA (US); Heidi Zhang, Los Gatos, CA (US); Patrick Heffernan, Campbell, CA (US)

Correspondence Address: THOMPSON HINE L.L.P., 2000 COURTHOUSE PLAZA , N.E., 10 WEST SECOND STREET, DAYTON, OH, 45402, US

(73) Assignee: MedDetect Inc., Los Gatos, CA

(21) Appl. No.: 10/205,599

(22) Filed: Jul. 25, 2002

(51) Int. Cl. A61B 10/00; A61B 5/00; G06F 17/00; G06N 5/02

(52) U.S. Cl. 600/300; 706/924; 128/922; 706/46

(57) ABSTRACT

A computer aided detection (CAD) system for assigning CAD applications to digital images. The system comprises a case input system (CIS), image data repositories and an algorithm server. There is also provided a method for assigning CAD applications to digital images the method comprising the steps of selecting an acquisition model (AM) and selecting a CAD application using the selected AM.

### CROSS-REFERENCE TO RELATED APPLICATIONS

This is the first application filed for the present invention.

### TECHNICAL FIELD

The invention relates to automated detection of features in digital images, and in particular, to a system and method for assigning a computer aided detection (CAD) application to a digital image.

### BACKGROUND OF THE INVENTION

Computer aided detection (CAD) is being used extensively to help health practioners by complementing traditional image diagnosis methods which rely on visual analysis.

CAD applications have been designed to analyze digitized images obtained from various imaging modalities such as X-ray, MRI, and ultrasound. Different applications are normally required to support the analysis of images acquired using different modalities. Furthermore, different applications may be required to properly analyse different images obtained using a given modality, due to differences in the properties of the images.

X-ray mammography is one type of diagnosis procedure amenable to CAD analysis. CAD analysis of X-ray films involves the digitization of the films and processing of the digitized mammograms with one or more CAD applications to produce a CAD report indicating areas of the breast that may exhibit abnormalities. The most common type of X-ray mammography exam is a screening exam which is performed on asymptomatic patients. Typically, several mammograms (taken at different viewing angles) are required to provide a set of images covering the entire volume of each breast. The standard practice in North America is to obtain 4 views; two views for each breast, which are taken along the cranio-caudal (CC) and medio-lateral oblique (MLO) planes, respectively. However, in special circumstances, such as with patients who have previously undergone mastectomy, the acquisition of a number of views that differs from the standard 4 views may be required. Special circumstances may also include examination of symptomatic patients, in which cases the radiologist may request additional views or views along axes that differ from the standard CC and MLO axes.

In addition to the modality of an image, the choice of the appropriate CAD application to analyze an image is dictated by: the type of analysis to be performed; the desired sensitivity; characteristics of the image or films; the anatomy and physiology of the subject being analyzed. In this regard, the anatomy and physiology of the patient may have been modified by previous chemical or surgical treatments thus requiring that different CAD applications be used.

Known CAD systems typically support a single imaging modality, and use a single CAD application for analyzing all the images. Other systems can support several imaging modalities and therefore use more than one CAD application. Such systems are normally designed to associate a given image with a specific CAD algorithm. For example, in U.S. Pat. No. 5,235,510 (Yamada et al.), a predetermined set of attributes (e.g. modality and subject) are stored in a table associated with each image. The attribute data stored in the table are used as search parameters to identify the proper CAD application. This system suffers from various limitations. For example, the same set of attributes are stored for each image. However, in some cases it may be desirable to store more (or at least different) attributes for some images, or some image modalities, than for others. Accordingly, a flexible technique for assigning CAD applications to digital images remains highly desirable.

### SUMMARY OF THE INVENTION

It is an object of the invention to provide a flexible technique for assigning CAD applications to digital images.

An aspect of the present invention provides a method for assigning a computer aided detection (CAD) application to a digital image. The method comprises the steps of: selecting an acquisition model from a plurality of acquisition models based on at least one attribute of the digital image; and selecting a CAD application from a plurality of CAD applications using the selected acquisition model.

In some embodiments, selection of an acquisition model may include the steps of: obtaining the at least one attribute of the digital image, and performing a search to identify the acquisition model using the obtained attributes. The CAD application may be selected by obtaining information concerning the digital image in accordance with the selected acquisition model and performing a search to identify the CAD application using at least the obtained information.

The digital image may be acquired using any suitable imaging modality including, but not limited to: X-ray imaging, ultrasound imaging, magnetic resonance imaging (MRI), computer tomography (CT) and nuclear medicine. The subject of the image may, for example, include a portion of a patient's body.

Another aspect of the present invention provides a system for assigning a computer aided detection (CAD) application to a digital image. The system includes a case input system for selecting an acquisition model from a plurality of acquisition models, based on at least one attribute of the digital image, and for selecting a CAD application from a plurality of CAD applications using the selected acquisition model.

The system may further include an algorithm server for retrieving and applying the selected CAD application to the digital image to produce CAD results.

### BRIEF DESCRIPTION OF THE DRAWINGS

Further features and advantages of the present invention will become apparent from the following detailed description, taken in combination with the appended drawings, in which:

FIG. 1 is a diagram of an embodiment of the system of the instant invention;

FIGS. 2a and 2b schematically illustrate respective formats of acquisition models for X-ray and ultrasound mammography;

FIG. 3 is a schematic representation of the acquisition model selection process; and

FIG. 4 is a schematic representation of the CAD application selection process.

### DETAILED DESCRIPTION OF THE PREFERRED EMBODIMENT

The present invention provides a method and a system for assigning a CAD application to a digital image.

In the following description, features of the invention are described by way of an exemplary embodiment that is optimized for mammography. However, it will be understood that the present invention is in no way limited to such embodiment. In fact, the present invention can equally be applied to image analysis and computer detection of a wide range of subjects, utilizing images obtained by any suitable modality.

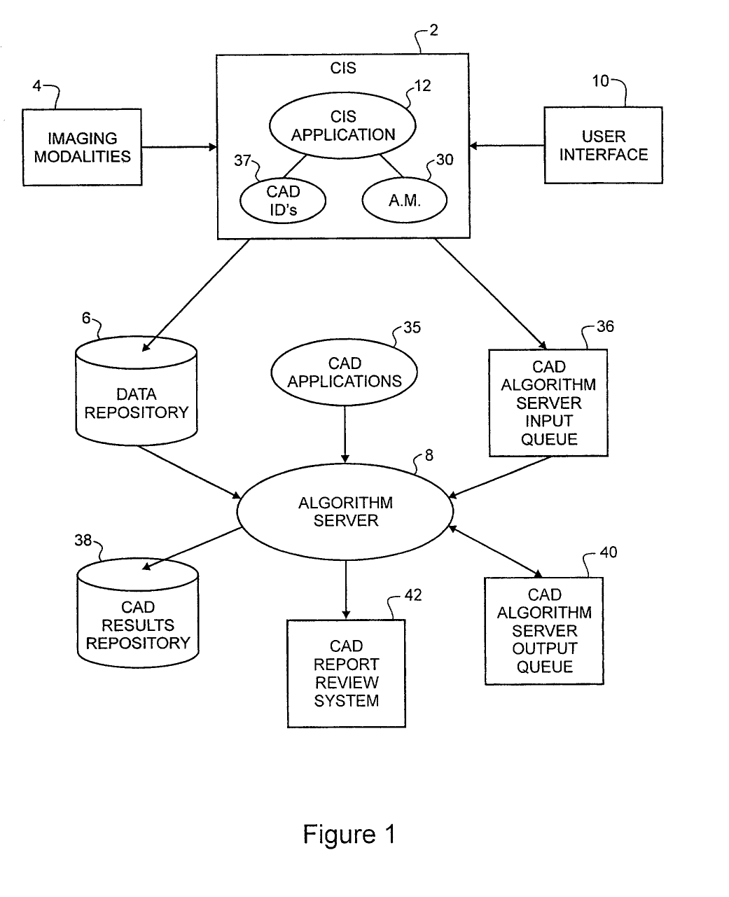

An embodiment of the system used to implement the method of the invention is schematically represented in FIG. 1. The system generally comprises a Case Input System (CIS) 2 that is directly or indirectly linked to one or more imaging modalities 4 which may in turn include a suitable image digitizing device (not shown), such as an X-ray film digitizer, to produce digitized images. The imaging modalities include but are not limited to: X-ray imaging, ultrasound, magnetic resonance imaging (MRI), computer tomography (CT) and nuclear medicine. The CIS 2 acquires the digitized images and may perform quality control, for example to determine whether certain characteristics of the digital images, such as their orientation and completeness, are adequate for the images to be processed by CAD applications. The CIS can also be linked to a data repository 6 to store the acquired digital images; an algorithm server 8; and a user interface 10 such as a key board or a touch screen to enable a user (not shown) to enter instructions pertaining to the identification and processing of the films or to otherwise interact with the system. The CIS also includes CIS applications 12 for properly identifying and transferring the digital images.

In the illustrated embodiment, digitized images from the CIS are stored in a repository 6 that is accessible to the algorithm server 8. Alternatively, the images can be stored in an archive such as a conventional Digital Imaging and Communication in Medicine (DICOM) archive, which can also be accessible to the algorithm server. Digitized images can also be stored to a repository that is accessible to an image review system.

Images belonging to the same patient and acquired as part of a prescribed examination, for example an X-ray mammography screening exam, can be grouped together to form a case. A case may comprise one or several images. The process of assigning a CAD application can thus be applied to a case instead of individual images. However, it will be appreciated that CAD applications may also be assigned to individual images within the case if the latter comprises more than one image.

The digital images are preferably identified with a unique identifier such that any digitized image is distinguishable from any other digitized image including those belonging to the same case. Furthermore the images in a case are preferably identified with an identifier common to all images belonging to a given case.

In general, a CAD application is assigned to a digital image by selecting an acquisition model (AM) based on one or more attributes of the digital image; and then selecting the most appropriate CAD application using information contained in the selected AM. The image attributes used to select an AM are preferably attributes common to a broad range of images, such as, for example, the modality. However, other image attributes may be used as desired. Preferably, at least one AM will be provided for each modality supported by the system. In cases where more than one AM is provided for a particular modality, additional attributes, such as the subject may be used to select the appropriate AM.

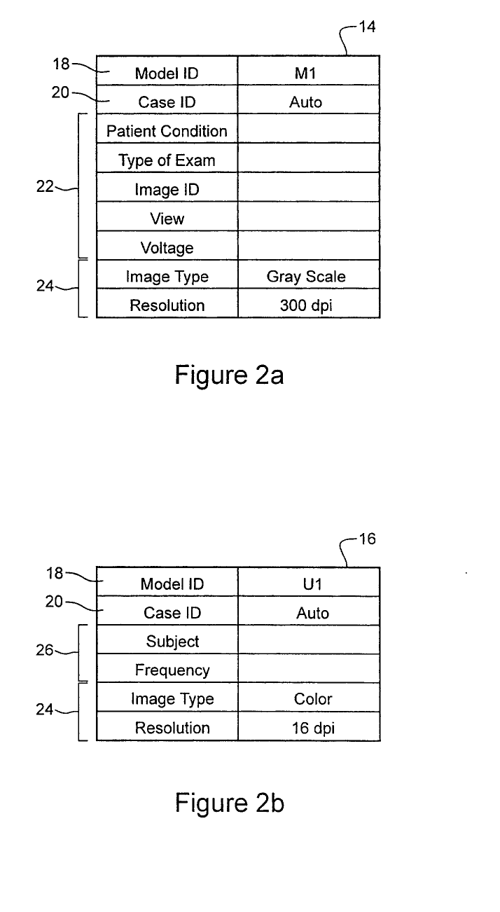

In general, an AM is a data table for storing relevant information pertaining to a set of one or more related images. As such, each AM will normally contain a respective different number of fields, and field contents. For example, FIGS. 2a and 2b illustrate exemplary acquisition models for X-ray 14 and ultrasound mammography 16, respectively. In the AM of FIG. 2a, header fields 18 are provided for the predetermined acquisition model identifier, and a case identifier 20 assigned by the CIS system. In addition, a set of case parameter fields 22 are provided for storing user input data concerning the patient condition (e.g. symptomatic/asymptomatic); type of exam (screening/diagnostic); image identifier and view, and the X-ray machine voltage used to develop the image. Finally, a set of default case parameters 24 (e.g. image type and resolution) can also be included. These default parameters can be preselected based on known properties of the imaging modality and image digitizing system, and thus do not need to be input by a user for each case. As shown in FIG. 2b, for ultrasound mammography, fewer and different case parameters 26 may be needed in order to select the appropriate CAD, and this is reflected in the format of the AM. As will be appreciated, the format of each AM can be chosen, as desired, to contain relevant information concerning each case and so facilitate selection of the appropriate CAD application. Thus more, or fewer fields may be contained in each AM, and these fields may be the same, or different from those illustrated in FIG. 2 as desired.

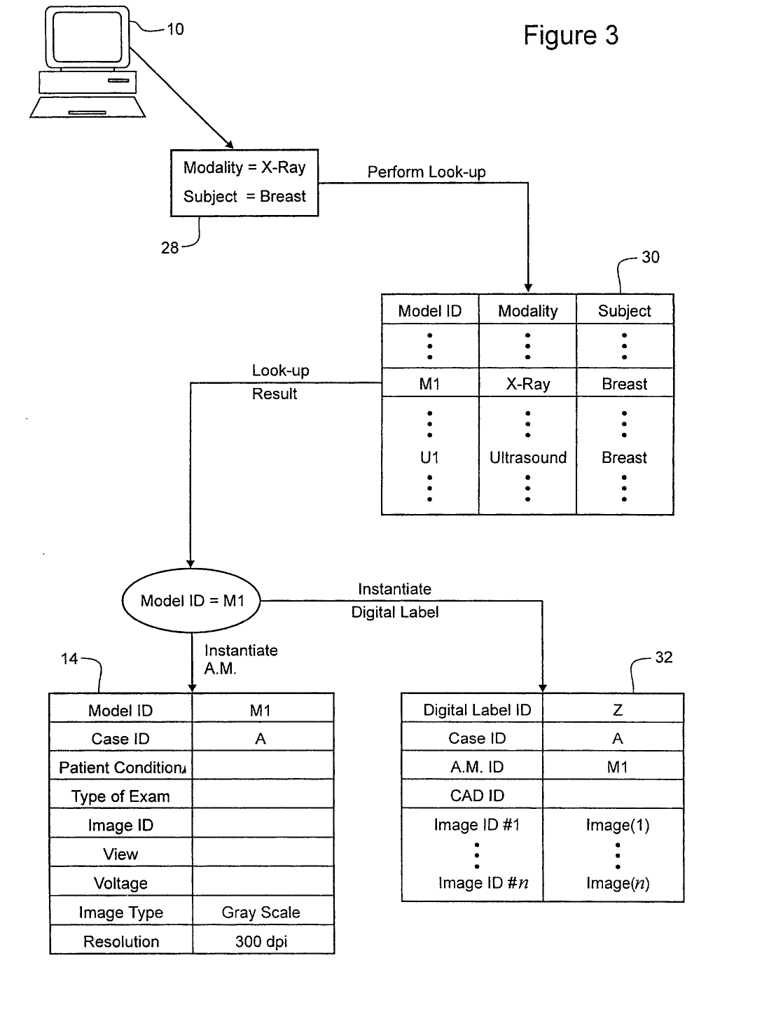

FIG. 3 illustrates principle steps in an exemplary process for selecting an AM. As mentioned above, an AM is selected based on a predetermined set of one or more image attributes, such as the modality and/or subject. This information may be known in advance, or input by a user at a time of image acquisition. Various methods may be employed to implement selection of the AM. In the example of FIG. 3, image attributes 28 obtained through the user interface, are used to access a AM look-up-table 30 to obtain the Model I.D. of the appropriate AM. Using the Model I.D., the CIS application can then instantiate the selected AM 14, and associate it with the image. Instantiation of the AM in this manner can result in the generation of suitable user-prompts to obtain input of the required case parameters, and confirmation of default parameter values. If desired, the user can be prompted for further information, based on their input of initial case parameters. For example, if the user indicates that the AM applies to more than one image, the list of case parameters can be automatically increased to provide suitable parameter fields for each image. Thus the AM of the present invention may not be statically defined, but may dynamically expand (or contact) based on a user's input. Association of the selected AM with the image can conveniently be accomplished by instantiating a primary digital label 32, for storing the model I.D. and image I.D. This arrangement has the advantage that the AM and the associated image(s) can be stored separately without loosing information concerning both the model and the images(s).

One or more different CAD applications may be used to analyze images obtained using a given imaging modality. Therefore, the selected AM will frequently be associated with more than one CAD application. In effect, the selection of the AM identifies a set of candidate CAD applications 34, from among the CAD applications 35 supported by the system, that can potentially be used to analyze images having the attributes provided by the user. Selection of the appropriate CAD application, from among the candidate applications, is accomplished using the case parameters input by the user following selection of the AM.

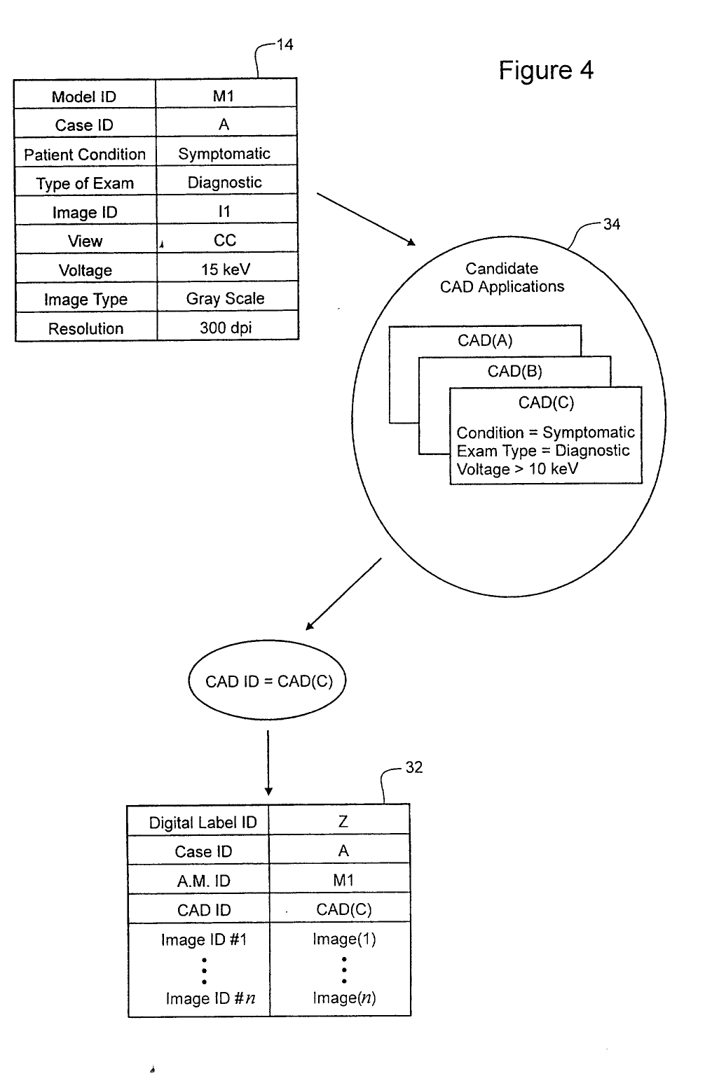

FIG. 4 illustrates principle steps in an exemplary process selecting the CAD application. In the example of FIG. 4, each CAD application is identified by a CAD I.D., (the CAD ID's 37 being stored in the CIS as shown in FIG. 1) and is associated with a predetermined set of selection criteria, which preferably follow the case parameter fields of the AM. Thus, the case parameter values input by the user, (and stored in the AM) can be compared with the selection criteria of each candidate CAD application. The CAD application having selection criteria that most closely matches the case parameter values stored in the AM is then selected for analyzing the image. In this case, the CAD I.D. identifying the selected CAD application is inserted into the primary digital label 32.

In the above examples, the entry of attributes and case parameters is effected by the user through user interface 10. Alternatively, some or all of these values may be automatically entered using automatic reading of the information. In the case of X-ray films for example, a bar code label containing the required information may be affixed to the film and scanned when the film is digitized. In a further alternative, one or more attributes and case parameters may be automatically determined using an algorithm capable of analyzing selected features of the original image, such as an X-ray film, or the corresponding digitized image.

Once a CAD application ID has been assigned to a digital image, the primary digital label identifying the selected AM, CAD application, and the associated images can be inserted into a CAD algorithm server input queue 36. The algorithm server 8 can then read the label from the queue, and execute the appropriate CAD application to process the image.

The processing of the image by a CAD application typically allows the identification of regions of interest (ROI) in the image. In mammograms, these ROI may comprise regions of the breast exhibiting abnormalities. The actual results of the CAD processing indicate the location and the nature of any abnormalities. The CAD results can be stored in a repository 38 and a secondary digital label, identifying the CAD results, associated with the image and inserted in a CAD algorithm server output queue 40. The algorithm server can read the secondary label from the queue in order to execute an appropriate CAD result report application, that will produce a CAD report that can be displayed or printed at a CAD report review system 42.

If desired, the AM may also include instructions specifying an appropriate format for the CAD report, since the desired format of the CAD report may vary depending, inter alia, on the number of views, the type of CAD algorithm used for the analysis and on the information requested by the user.

The present invention has been described with regards to the preferred embodiments, however it will be obvious to a person skilled in the art that a number of modifications and variations can be made without departing from the scope of invention as described herein. The scope of the invention is therefore intended to be limited solely by the scope of the appended claims.

I/we claim:

1. A method for assigning a computer aided detection (CAD) application to a digital image the method comprising steps of:
a) selecting an acquisition model from a plurality of acquisition models based on one or more attributes of the digital image; and
b) selecting a CAD application from a plurality of CAD applications using the selected acquisition model.

2. A method as claimed in claim 1 wherein the step of selecting an acquisition model comprises steps of:
a) obtaining the attributes of the digital image; and
b) performing a search to identify the acquisition model that most closely matches the obtained attributes of the digital image.

3. A method as claimed in claim 2 wherein the attributes comprises any one or more of: a modality of the image acquisition and a subject of the image.

4. A method as claimed in claim 3 wherein the modality of image acquisition comprises anyone or more of: X-ray imaging, ultrasound imaging, magnetic resonance imaging (MRI), computer tomography (CT).

5. A method as claimed in claim 3 wherein the subject of the image comprises anyone or more of: breast, chest, heart, head, lungs and bones.

6. A method as claimed in claim 1 wherein the step of selecting the CAD application comprises steps of:
a) obtaining information concerning the digital image in accordance with the selected acquisition model;
b) performing a search to identify the CAD application that most closely matches the obtained information.

7. A method as claimed in claim 6 wherein the information comprises case parameters.

8. A method as claimed in claim 7 wherein the case parameters comprise anyone or more of: X-ray film acquisition voltage, ultrasound frequency, number of images and type of exam.

9. A method as claimed in claim 8 wherein the type of exam comprises any one or more of: a screening exam and a diagnostic exam.

10. A method as claimed in claim 1 further comprising the step of associating a primary digital label, containing information identifying the selected acquisition model and/or CAD application, with the digital image.

11. A method as claimed in claim 10 further comprising the step of inserting the primary digital label in a CAD algorithm server input queue.

12. A method as claimed in claim 11 further comprising steps of:
a) reading the digital label in the CAD algorithm server input queue;
b) obtaining the selected CAD application; and
c) processing the digital image with the selected CAD algorithm to produce CAD results.

13. A method as claimed in claim 12 further comprising the step of associating a secondary digital label, containing information identifying the CAD results, with the digital image.

14. A method as claimed in claim 13 further comprising the step of inserting the secondary digital label in a CAD algorithm server output queue.

15. A system for assigning a computer aided detection (CAD) application to a digital image the system comprising a case input system for selecting an acquisition model from a plurality of acquisition models based on at least one attribute of the digital image and for selecting a CAD application from a plurality of CAD applications using the selected acquisition model.

16. A system as claimed in claim 15 wherein the case input system comprises:
a) a user interface for obtaining the at least one attribute of the digital image and for obtaining information concerning the digital image in accordance with the selected acquisition model; and
b) searching applications for performing searches to identify the acquisition model using the at least one attribute of the digital image and to identify the CAD application using at least the obtained information concerning the digital image.

17. A system as claimed in claim 15 further comprising an algorithm server for retrieving and applying the selected CAD algorithm to the digital image to produce CAD results.

18. A system as claimed in claim 17 further comprising a digital label identifier for associating a primary and a secondary digital label with the digital image.

19. A system as claimed in claim 18 further comprising a CAD algorithm server input queue and a CAD algorithm output queue for storing the primary and secondary digital labels respectively.

20. A system as claimed in claim 15 further comprising a repository for storing the digital image and the CAD results.

21. A system as claimed in claim 20 further comprising a CAD report generator to display CAD results.
